(19) United States
(12) Patent Application Publication
(10) Pub. No.: US 2005/0142078 A1
(43) Pub. Date: Jun. 30, 2005

(54) Method of stimulation of melanin production and induction of skin tanning

(76) Inventors: Robert T Dorr, Tucson, AZ (US); Norman Levine, Tucson, AZ (US); Gregory Ertl, Yuma, AZ (US); David S. Alberts, Tucson, AZ (US)

Correspondence Address: GREENLEE WINNER AND SULLIVAN P C, 4875 PEARL EAST CIRCLE, SUITE 200, BOULDER, CO, 80301, US

(21) Appl. No.: 10/504,492

(22) PCT Filed: Feb. 25, 2003
(86) PCT No.: PCT/AU03/00230

Related U.S. Application Data
(60) Provisional application No. 60/359,735, filed on Feb. 26, 2002.

(51) Int. Cl. A61K 38/08; A61K 7/42

(52) U.S. Cl. 424/59; 514/17

(57) ABSTRACT

A method of stimulating the production of melanin by the pigment-producing cells (keratinocytes and/or melanocytes) of the skin, in particular for the induction of skin tanning in humans, comprises administering alpha-MSH or an alpha-MSH analogue and exposing the skin to ultraviolet irradiation.

Partial funding for the research leading to the present invention was received from the National Institute of Health. Accordingly, the Government of the United States of America retains certain rights in the invention described herein.

### FIELD OF THE INVENTION

The present invention relates broadly to a method of stimulating the production of melanins by the pigment-producing cells (keratinocytes and/or melanocytes) of the skin, and in particular to a method for the induction of skin tanning in humans.

### BACKGROUND OF THE INVENTION

The melanocortins (also referred to as melanotropin) include a family of peptide hormones that induce pigmentation by interaction with melanocortin 1 receptors (MC1R) in the epidermis¹. The primary pigmentary hormone that is released from the pars intermedia of the pituitary gland in some non-human animals, and from UV-B exposed keratinocytes in human skin, is alpha Melanocyte Stimulating Hormone (alpha-MSH)¹. This 13 amino acid peptide binds to MC1R to induce cyclic AMP-mediated signal transduction leading to the synthesis of melanin polymers from DOPA precursors¹. Two type of melanins can be expressed in humans. The brownish-black pigment eumelanin is believed to convey protection from sun damage, whereas the reddish, sulfur-containing pigment, pheomelanin is often expressed in light-skinned human populations that report a poor tanning response to sunlight². These poorly-tanning, easily-burning populations, termed Type 1-2 by Fitzpatrick scale³, may possess defects in the MC1R gene⁴, and are generally thought to be at a greater risk of developing skin cancers^{5,6}.

It has previously been disclosed that a super-potent derivative of alpha-MSH, melanotan-1, (Nle⁴-D-Phe⁷-alpha MSH), can induce tanning in human volunteers with such poorly-tanning skin types, but especially in subjects with easily tanning skin types, (Fitzpatrick scale 3-4)⁷. Melanotan-1 (MT-1), contains two amino acid substitutions and is approximately 100 to 1,000-fold more potent than the native hormone at inducing pigmentation in experimental systems such as the frog skin bioassay⁸ or in cultured human keratinocytes⁸. In humans, MT-1 primarily induces eumelanin synthesis in the skin in concert with it's tanning effect⁹. Although melanotropins have been postulated to effect immunologic changes¹⁰⁻¹², all of the prior trials reported only minimal side effects such as facial flushing and transient GI upset, unless doses greater than those needed for tanning were administered¹³.

U.S. Pat. No. 4,457,864 (issued Jul. 3, 1984), discloses analogues of alpha-MSH, including Nle⁴-D-Phe⁷-alpha MSH. Cyclic analogues of alpha-MSH are disclosed in U.S. Pat. No. 4,485,039 (issued Nov. 27, 1984). The use of these and other analogues of alpha-MSH for stimulating the production of melanin by integumental melanocytes is disclosed in Australian Patent No. 597630 (dated Jan. 23, 1987) and U.S. Pat. Nos. 4,866,038 (issued Sep. 12, 1989), 4,918,055 (issued Apr. 17, 1990) and 5,049,547 (issued Sep. 17, 1991). Australian Patent No. 618733 (dated May 20, 1988), and U.S. Pat. Nos. 5,674,839 (issued Oct. 7, 1997) and 5,714,576 (issued Feb. 3, 1998) disclose further linear and cyclic alpha-MSH fragment analogues, and the use of these biologically-active analogues in stimulating melanocytes. The contents of all these published Australian and U.S. patents are incorporated herein by reference (see also refs.^{25,26}).

All of the previously-reported clinical trials with MT-1 were performed in human volunteers who were instructed to avoid sunlight and use sunscreens with an SPF of 30 to apply to all sun-exposed skin sites^{7,9,13}. Thus, the effect in humans of MT-1 when combined with either sunlight or simulated UV radiation, has not been tested or reported otherwise.

In work leading to the present invention, the inventors have carried out clinical trials in human volunteers with MT-1 combined with either direct sunlight, or with small doses of UV-B radiation delivered from a solar simulator. The intent of these studies was to examine the effect of MT-1 on skin tanning in humans, and in particular, whether there was any evidence of additive pigmentation or an alteration in the response of skin to UV-B, measured by the presence of sunburn cells. In addition, a subset of patients receiving MT-1 underwent detailed analysis of 17 different B- and T-lymphocyte sub-populations to evaluate the effect of MT-1 on immunologic status.

As a result of these clinical trials, the inventors have discovered that the combined use of a melanotropic peptide such as MT-1 and UV radiation results in unexpected levels of skin tanning and prolonged retention of pigmentation. Accordingly, the methods of the present invention enable enhanced skin pigmentation from sunlight exposure, reduction in the amount of sunlight exposure required for visually-apparent skin tanning, safe acceleration of the production of sun-protective skin tanning, and reduction of sun-induced skin damage by rapid induction of long-lasting eumelanin expression in sun-exposed areas.

### SUMMARY OF THE INVENTION

Bibliographic details of the publications referred to in this specification by reference number are collected at the end of the specification.

Throughout this specification, unless the context requires otherwise, the word “comprise”, or variations such as “comprises” or “comprising”, will be understood to imply the inclusion of a stated integer or step or group of integers or steps but not the exclusion of any other integer or step or group of integers or steps.

Those skilled in the art will appreciate that the invention described herein is susceptible to variations and modifications other than those specifically described. It is to be understood that the invention includes all such variations and modifications, the invention also includes all of the steps, features, compositions and compounds referred to or indicated in this specification, individually or collectively, and any and all combinations or any two or more of said steps or features.

In one aspect, the present invention provides a method for the stimulation of integumental melanocytes in a mammal, which comprises the steps of:
(i) administering to said mammal an amount of alpha-MSH or an alpha-MSH analogue effective to stimulate melanocytes in the skin or other epidermal tissue; and
(ii) exposing said skin or other epidermal tissue to ultraviolet (UV) irradiation.

In another aspect, the present invention provides a method for stimulating melanin production in a mammal, which comprises the steps of:
(i) administering to said mammal an amount of alpha-MSH or an alpha-MSH analogue effective to stimulate melanin production in the skin or other epidermal tissue; and
(ii) exposing said skin or other epidermal tissue to ultraviolet (UV) irradiation.

In yet another aspect, the present invention provides a method for inducing tanning in a mammal, which comprises the steps of:
(i) administering to said mammal an amount of alpha-MSH or an alpha-MSH analogue effective to induce tanning in the skin or other epidermal tissue; and
(ii) exposing said skin or other epidermal tissue to ultraviolet (UV) irradiation.

The present invention further extends to the use of alpha-MSH or an alpha-MSH analogue in a method for the stimulation of integumental melanocytes in a mammal, more particularly for stimulating melanin production, and even more particularly for inducing tanning in a mammal. In this aspect, the invention extends in particular to the use of alpha-MSH or an alpha-MSH analogue in a method for inducing skin tanning in a human.

### DETAILED DESCRIPTION OF THE INVENTION

As described above, the present invention provides a method for the stimulation of integumental melanocytes in a mammal, which comprises the steps of:
(i) administering to said mammal an amount of alpha-MSH or an alpha-MSH analogue effective to stimulate melanocytes in the skin or other epidermal tissue; and
(ii) exposing said skin or other epidermal tissue to ultraviolet (UV) irradiation.

The present invention also extends to a method for stimulating melanin production in a mammal. Preferably, the mammal is a human, however the methods of the present invention also extend to other mammals in which increased melanin production may be desired, for example to change coat (hair) coloration. In particular, the invention relates to a method for inducing tanning in a mammal, more particularly, for inducing skin tanning in a human.

The step of exposing the skin or other epidermal tissue to UV irradiation may be carried out simultaneously with, or subsequent, to the step of administering the alpha-MSH or alpha-MSH analogue to the skin or other epidermal tissue. Preferably, the step of exposing to UV irradiation is carried out subsequent to administration of the alpha-MSH or alpha-MSH analogue.

The step of exposure to UV irradiation may be performed either by exposure to artificial UV-B and/or UV-A irradiation from a solar simulator or similar UV source, or preferably, by exposure to natural sunlight. Preferably, the UV irradiation consists of or comprises UV-B irradiation.

Alpha-MSH analogues suitable for use in the method of the present invention include those disclosed in U.S. Pat. Nos. 4,457,864, 4,485,039, 4,866,038, 4,918,055, 5,049,547, 5,674,839 and 5,714,576 and Australian Patents Nos. 597630 and 618733, and the disclosure of each of these patent documents is incorporated herein by reference (see also refs.^{25,26}).

In its broadest aspects, the present invention extends to the use of any of these alpha-MSH analogues. These analogues may be synthesised according to the procedures set out in these patent documents or other references, or according to methods used in preparing synthetic alpha-MSH which are well-known to persons skilled in this art, for example, by solid phase peptide synthesis.

Suitable alpha-MSH analogues for use in accordance with the present invention include compounds of the formula:
R₁-W-X-Y-Z-R₂
wherein
R¹ is selected from the group consisting of Ac-Gly-, Ac-Met-Glu-, Ac-Nle-Glu-, and Ac-Tyr-Glu-;
W is selected from the group consisting of -His- and -D-His-;
X is selected from the group consisting of -Phe-, -D-Phe-, -Tyr-, -D-Tyr-, -(pNO₂)D-Phe⁷-;
Y is selected from the group consisting of -Arg- and -D-Arg-;
Z is selected from the group consisting of -Trp- and -D-Trp-; and
R₂ is selected from the group consisting of —NH₂; -Gly—NH₂; and -Gly-Lys—NH₂.

As used hereinabove and below, Ala=alanine, Arg=arginine, Glu=glutamic acid, Gly=glycine, His=histidine, Lys=lysine, Met=methionine, Nle=norleucine, Phe=phenylalanine, (pNO₂)Phe=paranitrophenylalanine, Plg=phenylglycine, Pro=proline, Ser=serine, Trp=tryptophan, TrpFor=N¹-formyl-tryptophan, Tyr=tyrosine, Val=valine. All peptides are written with the acyl-terminal end at the left and the amino terminal end to the right; the prefix “D” before an amino acid designates the D-isomer configuration, and unless specifically designated otherwise, all amino acids are in the L-isomer configuration.

Compounds suitable for use in the present invention include:
[D-Phe⁷]-alpha-MSH
[Nle⁴, D-Phe⁷]-alpha-MSH
[D-Ser¹, D-Phe⁷]-alpha-MSH
[D-Tyr², D-Phe⁷]-alpha-MSH
[D-Ser³, D-Phe⁷]-alpha-MSH
[D-Met⁴, D-Phe⁷]-alpha-MSH
[D-Glu⁵, D-Phe⁷]-alpha-MSH
[D-His⁶, D-Phe⁷]-alpha-MSH
[D-Phe⁷, D-Arg⁸]-alpha-MSH
[D-Phe⁷, D-Trp⁹]-alpha-MSH
[D-Phe⁷, D-Lys¹¹]-alpha-MSH
[D-Phe-⁷, D-Pro¹²]-alpha-MSH
[D-Phe⁷, D-Val¹³]-alpha-MSH
[D-Ser¹, Nle⁴; D-Phe⁷]-alpha-MSH
[D-Tyr², Nle⁴, D-Phe⁷]-alpha-MSH
[D-Ser³, Nle⁴, D-Phe⁷]-alpha-MSH
[Nle⁴, D-Glu⁵, D-Phe⁷]-alpha-MSH
[Nle⁴, D-His⁶, D-Phe⁷]-alpha-MSH
[Nle⁴, D-Phe⁷, D-Arg⁸]-alpha-MSH
[Nle⁴, D-Phe⁷, D-Trp⁹]-alpha-MSH
[Nle⁴, D-Phe⁷, D-Lys¹¹]-alpha-MSH
[Nle⁴, D-Phe⁷, D-Pro¹²]-alpha-MSH
[Nle⁴, D-Phe⁷, D-Val¹³]-alpha-MSH
c[Cys⁴, Cys¹⁰]-alpha-MSH
c[Cys⁴, D-Phe⁷, Cys¹⁰]-alpha-MSH
c[Cys⁴, Cys¹¹]-alpha-MSH
c[Cys⁵, Cys¹⁰]-alpha-MSH
c[Cys⁵, Cys¹¹]-alpha-MSH
c[Cys⁴, Cys¹⁰]-alpha-MSH₄₋₁₃
c[Cys⁴, Cys¹⁰]-alpha-MSH₄₋₁₂
[Nle⁴, D-Phe⁷]-alpha-MSH₄₋₁₀
[Nle⁴, D-Phe⁷]-alpha-MSH₄₋₁₁
[D-Phe⁷]-alpha-MSH₅₋₁₁
[Nle⁴, D-Tyr⁷]-alpha-MSH₄₋₁₁
[(pNO₂)D-Phe⁷]-alpha-MSH₄₋₁₁
[Tyr⁴, D-Phe⁷]-alpha-MSH₄₋₁₀
[Tyr⁴, D-Phe⁷]-alpha-MSH₄₋₁₁
[Nle⁴]-alpha-MSH₄₋₁₁
[Nle⁴, (pNO₂)D-Phe⁷]-alpha-MSH₄₋₁₁
[Nle⁴, D-His⁶]-alpha-MSH₄₋₁₁
[Nle⁴, D-His⁶, D-Phe⁷]-alpha-MSH₄₋₁₁
[Nle⁴, D-Arg⁸]-alpha-MSH₄₋₁₁
[Nle⁴, D-Trp⁹]-alpha-MSH₄₋₁₁
[Nle⁴, D-Phe⁷, D-Trp⁹]alpha-MSH₄₋₁₁
[Nle⁴, D-Phe⁷]-alpha-MSH₄₋₉
[Nle⁴, D-Phe⁷, D-Trp⁹]-alpha-MSH₄₋₉

Preferred compounds include:
[Nle⁴, D-Phe⁷]-alpha-MSH
[Nle⁴, D-Phe⁷]-alpha-MSH₄₋₁₀
[Nle⁴, D-Phe⁷]-alpha-MSH₄₋₁₁
[Nle⁴, D-Phe⁷, D-Trp⁹]-alpha-MSH₄₋₁₁
[Nle⁴, D-Phe⁷]-alpha-MSH₄₋₉

The most preferred alpha-MSH analogue for use in the methods of this invention is [Nle⁴, , D-Phe⁷]-alpha-MSH, referred to hereinafter as “melanotan-1” or “MT-1”.

The compounds useful in this invention may be administered by a variety of routes including oral, parenteral or transdermal. The term “parenteral” is used herein to encompass any method by which the compounds according to the present invention are introduced into the systemic circulation and include intravenous, intramuscular and subcutaneous injections. The term “transdermal” as used herein encompasses the administration of the compound by topical methods such as buccal or skin patches, intranasal or tracheal sprays, by solution for use as ocular drops, by suppositories for vaginal or anal routes of administration or by conventional topical preparations such as creams or gels for localised percutaneous delivery.

The compounds will be formulated in suitable compositions determined by the intended means of administration, according to methods and procedures well-known to those skilled in the art (see, for example, Remington's Pharmaceutical Sciences, 18th Edition, Mack Publishing Company, Pennsylvania, USA). For example, the compounds suitable for use in this invention may be formulated or compounded into pharmaceutical compositions comprising at least one compound of the present invention (the compositions may comprise one compound or admixtures of compounds according to the present invention) in admixture with a solid or liquid pharmaceutical excipient such as a diluent or carrier for oral or parenteral administration. As injection medium, water containing the usual pharmaceutical additives for injection solutions, such as stabilising agents, solubilising agents, and buffers is preferred. Among additives of this type are, for example, tartrate and citrate buffers, ethanol, complex forming agents such as ethylenediamine-tetraacetic acid, and high molecular weight polymers such as liquid polyethylene oxide for viscosity regulation. Solid carrier materials include, for example, starch, lactose, mannitol, methyl cellulose, talc, highly dispersed silicic acid, high molecular weight fatty acids such as stearic acid, gelatine, agar-agar, calcium phosphate, magnesium stearate, animal and vegetable fats, and high molecular weight polymers such as polyethylene glycols. Compositions suitable for oral administration can, if desired, contain flavouring and/or sweetening agents. For topical administration, the compounds may be preferably used with various conventional bases for topical preparations such as creams, ointments, gels, lotions or sprays, depending upon the desired mode of delivery of the ingredients to an individual. In manufacturing these preparations, the composition may also be mixed with conventional inert excipients such as thickening agents, emollients, surfactants, pigments, perfumes, preservatives, fillers and emulsifiers, all of which are well known and conventionally used in the formulation of transdermal or other preparations. Typically, these non-active ingredients will make up the greater part of the final preparation. Preferably, the compositions are manufactured to allow for controlled and/or sustained-release delivery.

The actual amount of administered compound according to the present invention may vary between fairly wide ranges depending upon the mode of administration, the excipients used, and the degree of stimulation desired. Such amounts are well within the skill of the pharmaceutical scientist to determine, and the amount administered to the mammal may be any amount chosen to stimulate melanotropic activity, for example, by formulation as an implant using poly (D, L lactide-co-glycolide polymer²⁴ or a similar biodegradable, biocompatible polymer as carrier.

In the work leading to the present invention, described in detail in the Example below, two clinical trials of a superpotent melanotropic peptide, melanotan-1 (MT-1), were performed in normal human volunteers with tanning skin types 3-4 (Fitzpatrick scale). The first study in 12 subjects used 0.16 mg/kg/day for 10 days plus UV-B radiation to the buttock to evaluate tanning synergy. The results show significant tanning in the MT-1 treated subjects, and especially at the UV-B-irradiated buttock skin sites. Immunologic parameters were unaltered in 7 of these subjects. A second study randomized subjects to placebo, plus 3-5 days of sunlight to the back, (n=3), or sunlight plus MT-1 at 0.16 mg/kg/day×20 days over 4 weeks, (n=5). There was significant whole-body tanning in the MT-1 group and the back areas required 50% less sunlight for equivalent tanning. In addition, tanning of the back area was maintained for over 3 months in the MT-1 treated group compared to 6 weeks in the controls. These results establish that MT-1 synergises with sunlight to produce a dark and long-lasting skin pigmentation.

### BRIEF DESCRIPTION OF THE DRAWINGS

FIG. 1 Shows results of a clinical trial and compares mean (n=3) reflectance changes on the back over 6 weeks for the control group (only sunlight) at the sun-exposed back (squares) or the opposite, non-exposed site, (circles). The reflectance values are luminance (solid symbols) and b- scale (blue-yellow) hue (open symbols).

FIG. 2 Shows results of a clinical trial and compares mean (n=3) reflectance changes on the back over 10 weeks for subjects receiving sunlight to the back at the start of MT-1 dosing. Tanning is indicated by reduced luminance (squares) and increased b-scale hue (circles) for the sun-exposed back site (solid symbols) and non-exposed back site (open symbols).

FIG. 3 Shows results of a clinical trial and compares mean reflectance changes on the back over 10 weeks for subjects (n=2) receiving sunlight to the back starting 1 week after finishing MT-1. Tanning is indicated by reduced luminance (squares) and increased b-scale hue (circles) for the sun-exposed back site (solid symbols), and the non-exposed back site (open symbols).

The present invention is further described by reference to the following non-limiting Example.

### EXAMPLE

Materials and Methods:

Design: Two clinical trials were performed from 1991 to 1994 at the University of Arizona Medical School campus to evaluate the response of human skin to MT-1 when combined with sunlight or simulated ultraviolet-B-range, (UV-B), radiation. Normal subjects with a tanning skin type by history were treated with subcutaneous Melanotan I, (MT-1), at 0.16 mg/kg/day for two to four weeks. The effects on skin pigmentation were evaluated by serial reflectance measurements at 8 anatomic sites, beginning before treatment, at the end of treatment and for up to 4 weeks after MT-1 treatment. Placebo controls were used for Protocol 2 (MT-1 plus mid-day sunlight to half of the back area). Protocol 1 randomized subjects to UV-B exposure on the buttock at either the start of MT-1 treatment (Group A), or immediately after finishing the ten-day course of MT-1 (Group B)

Subjects: Normal subjects were recruited from newspaper ads and were screened to have Type 3-4 skin by the Fitzpatrick scale³, and for the lack of any history of skin conditions, including skin cancers, dysplastic nevus syndrome or atypical moles. All subjects were required to have normal laboratory values as assessed by serial chemistry (SMAC-20), CBC and urinalysis. Any women must have tested negative for pregnancy and agreed to avoid becoming pregnant by means of active contraception. Additional lab tests that were required by the FDA to be monitored included serum cortisol, LH and FSH, which were measured before treatment and at the end of the two week treatment period.

Melanotan-I: Nle⁴-D-Phe⁷ alpha melanocyte stimulating hormone, (MT-1), was prepared by solid-phase chemistry under GMP conditions at Bachem Inc, Torrance Ca. The white powder was reconstituted in bacteriostatic sodium chloride for injection and tested negative for pyrogens by the Limulus amebocyte lysate (LAL) assay¹⁴. It was stored frozen prior to thawing immediately prior to subject injections. All doses were subcutaneously administered into the upper arm or thigh using a 25 gauge needle. The doses were calculated using actual body weight to deliver 0.16 mg/kg/day of MT-1 per day. MT-1 was administered daily on Monday to Friday, for two consecutive weeks in Protocol 1 (10 total injections), and for 4 weeks in Protocol 2 (20 total injections). The 0.16 mg/kg dose was used for these Protocols following a Phase I study in a small number of subjects that showed this was the maximally effective daily dose for tanning with minimal side effects⁹.

UV Radiation was delivered by means of a solar simulator for Protocol 1, or by a series of timed mid-day sun exposures in the months of March through June, for Protocol 2.

Endpoints for both protocols included pigmentation measured as skin reflectance at eight different anatomic body sites: the forehead, cheek, dorsal neck, inner forearm, scapula, abdomen, buttock, and anterior leg. Skin chromaticity was measured by light reflectance recorded on a Minolta CR200 Chromameter^{R} (Minolta Camera, Osaka, Japan). The reflected light is received and split into three fractions. Luminance (or L-scale), indicates relative brightness from black to white and decreases with tanning. There are two colour scales; the a-scale for yellow to red, which does not change with tanning, and the b-scale which indicates blue-yellow hue and increases with tanning⁵⁻¹⁷. For these studies, only L-scale and b-scale responses were recorded and stored on a portable computer. Eight measurements per anatomic site are performed. Each subject served as his own control and reflectance was serially measured: from baseline (pre-dosing), then at the end of dosing and for several weeks thereafter, usually until the reflectance values returned to the baseline level for each subject. All subjects had their minimal erythema dose, (MED), of UV-B radiation defined at the outset of each study. The MED was defined using a series of graded doses of UV-B delivered from a solar irradiation simulator (Model 600 Multiport^{R} Solar Ultraviolet Simulator, Solar Light Co., Philadelphia, Pa.).

A secondary biologic endpoint was evaluated in Protocol 1. Seventeen different immunologic parameters were evaluated by flow cytometry tests on blood samples drawn for 7 patients treated with MT-1 This involved the quantification of distinct immune function cell types in peripheral blood, including pan B- cells and several T-lymphocytes subsets: natural killer (NK)T-cells, lymphokine activated killer (LAK) cells, CD-8 (suppressor) and CD-4 (helper) cells, IL-2 receptor+(CD3) cells, transferrin receptor positive T-cells, and three classes of T-cells found in skin (CD4/CD4RP, LFA-3 and HECA452/CD3 T-cells). For these studies, blood was withdrawn by peripheral venipuncture at two pre-dosing times, again at the completion of the ten subcutaneous doses, and then ten days after completion of dosing, a time at which the tanning response is generally maximal. The white blood cell fraction was separated by Ficol^{R} centrifugation and different subtypes of lymphocytes and monocytes were detected and quantified by automated fluorescence-activated cell sorting (Facscan^{R}, Becton-Dickinson, San Jose, Calif.).

Statistical analyses were performed on the reflectance (tanning) data in both protocols using analysis of variance with a post-test, the Student Neuman Keuls test, for any significant (p<0.05) ANOVA differences. To be considered a significantly darkened site required statistical changes in both the L-scale and the b-scale, each at p<0.05 for ANOVA followed by the multiple range test. The cumulative exposure time of sunlight exposure to a given level of skin tanning in Protocol 2 was also compared using ANOVA. The immunologic parameters were compared at two baseline time points and then on the day of dose 5, the day of dose 10 and ten days after dosing ended. The 95% confidence intervals were analysed for the two baseline values, and then between the mean of those baselines and the other time points. The Bonferroni adjustment was used to correct the p-value for the multivariate tests, such that a significant difference would be 0.05/17 (the number of immunologic variables), or p<0.0029 for significance.

### RESULTS

### Protocol 1

Effect of Melanotan 0.16 mg/kg/day plus Five Daily Doses of UV-B Radiation at the Beginning or End of MT-1 Dosing Period

The goal of this study was three-fold: (1) determine the effect of tanning at double the dose of MT-1 from the prior trial; (2) evaluate whether there was additive tanning if UV-B radiation was given at the beginning or end of MT-1 dosing period; and, (3) evaluate immune function parameters in subjects given MT-1. There were 12 subjects with Fitzpatrick skin types 3-4 randomized to this study. Group A, (n=7), received UV-B radiation to the buttock on the first five days of MT-1 dosing. Group B, (n=5), received the same three dose levels of UV-B radiation to the buttock for five consecutive days, starting 3 days after the last dose of MT-1 was administered. An individual subject's MED was determined visually following graded UV-B exposure to the opposite buttock prior to MT-1 dosing. The MED was defined as the amount of radiation, from 15.75 to 42 mjoules/cm², that produced faint erythema with four distinct borders, measured 24 hours after UV-B dosing. When combined with MT-1, the UV-B radiation was delivered daily for five consecutive days to the buttock area at three solar simulator settings of 0.25, 0.50 and 0.75, representing 5.25 mjoules/cm², 10.5 mj/cm² and 15.75 mjoules/cm², respectively. The MT-1 doses for both groups were administered by subcutaneous injection into the upper arm on Monday-Friday, for two consecutive weeks. There was a total of ten doses, each delivering 0.16 mg/kg. Characteristics of each group are summarised in Table 1.

TABLE 1
Skin Type Characteristics for Subjects Receiving MT-1 0.16 mg/kg
Plus Five Days of UV-B Radiation to the Buttock

| Subject | | | | MED | Days of |
|---|---|---|---|---|---|
| Number | Sex | Age | Skin Type¹ | (mjoules) | UV-B² |
| 063 | F | 54 | IV | 31.5 | 1-5 |
| 067 | M | 24 | III | 21.0 | 1-5 |
| 069 | F | 52 | IV | 26.25 | 1-5 |
| 070 | F | 45 | III | 26.25 | 1-5 |
| 072 | F | 36 | III | 36.75 | 1-5 |
| 073 | F | 51 | IV | 31.5 | 1-5 |
| 078 | M | 29 | III | 26.25 | 1-5 |
| 065 | M | 41 | IV | 31.5 | 15-19 |
| 066 | M | 27 | III | 26.25 | 15-19 |
| 071 | F | 49 | III | 31.5 | 15-19 |
| 074 | M | 24 | IV | 26.25 | 15-19 |
| 077 | F | 40 | IV | 26.25 | 15-19 |

¹Fitzpatrick scale for tanning vs. burning by personal history
²Five daily doses of UV-B to the buttock at 0.25, 0.5 and 0.75 of MED radiation

Tanning Results for the eleven subjects are summarised in Table 2. There was significant skin darkening at some body site in all but one individual, indicating an overall response rate of 10/11 subjects. The non-responsive subject was No. 073 (see Table 1 for characteristics). The sites of significant skin pigmentation varied for different subjects, and the most responsive skin sites were the forehead, cheek and scapula with 6/11 subjects responding at each site. Curiously, the neck was much less responsive with only 3/11 subjects exhibiting significant changes in both luminance and b-scale reflectance values from baseline. The results in Table 2 differ from our previous studies in that non-responsive sites in the past, such as the buttock, abdomen and anterior leg, exhibited significant skin darkening in 4 or 5 of the total 11 subjects in this trial. Another difference is the prolonged duration of significant darkening. The results in Table 2 also show that most subjects had not returned to their baseline reflectance values at the final evaluation at week 6 (4 weeks after MT-1 dosing ended). Finally, there was significant enhancement for MT-1 and the five UV-B doses delivered to the buttock on both the early “A” schedule, or the later “B” schedule.

TABLE 2
Reflectance Changes in 11 Subjects Given
Melanotan-1 0.16 mg/kg/day for 10 Injections

| | Mean | | | | |
|---|---|---|---|---|---|
| | Reflectance | | | | |
| | at | Mean Change in Absolute Luminance | | | |
| Anatomic | Baseline | (No. Subjects Significantly Different)²; | | | |
| Site | Luminance¹ | Week 2 | Week 3 | Week 4 | Week 6 |
| Forehead | (61.59) | −2.47(5) | −2.85(5) | −2.21(6) | −1.72(4) |
| Cheek | (61.06) | −2.58(5) | −2.11(5) | −1.92(6) | −1.88(4) |
| Neck | (60.35) | −2.16(3) | −2.46(3) | −1.98(3) | −2.35(3) |
| Abdomen | (67.29) | −1.26(2) | −1.75(5) | −1.49(3) | −0.79(3) |
| Scapula | (64.30) | −0.9(6) | −0.43(4) | −0.67(4) | −0.97(4) |
| Buttock | (68.25) | −0.24(0) | −1.05(5) | −0.67(1) | −0.46(0) |
| Forearm | (65.59) | −1.68(4) | −1.19(5) | −1.32(5) | −1.22(3) |
| Leg | (64.67) | +0.55(0) | −0.34(4) | 0.04(1) | −0.32(3) |
| Anterior | | | | | |

¹Mean baseline luminance for all 11 subjects.
²Number of subjects (in parentheses) of total 11 showing a significant decrease in luminance and increase in b-scale (data not shown for b-scale differences).

Side effects in Protocol 1 were quantitatively and qualitatively similar to the previous studies^{7,9,13}. The most common side effect was flushing of the upper body that occurred variably within minutes after MT-1 injection. About half of the subjects experienced a median of three instances of this self-limited reaction at some time during the two week dosing period. These reactions lasted from a few minutes up to an hour and were not associated with other sequelae. A mild sensation of nausea was reported in 4 of the 11 subjects. This effect was typically noted after the second or third injection of MT-1 and lasted for 30 minutes up to several hours. Because of the mild severity, antiemetic therapy was not required in any subject, but a few subjects described mild anorexia late in the evening on injection days. Fatigue was also reported in about one-third of the subjects, usually in the afternoon of the day of injection. This was variable in intensity, but was never severe enough to require bed rest. And, like the flushing reactions, the episodes of afternoon fatigue did not recur or increase in intensity with successive doses.

Immunologic Findings: Seven subjects in this protocol had five blood samples collected before, during and after receiving MT-1 to determine whether the acute drug regimen induced changes in 17 different types of white blood cells. The first two samples were baselines, drawn prior to dosing, about 8 weeks apart and were compared statistically. This analysis showed that for all 17 parameters in the two baselines, zero was contained in the 95% confidence interval. The average of these two baselines was then calculated and used for comparison to the other three time points: on the day of doses 5 and 10, and ten days thereafter. The results show that two parameters, T-memory cells (p=0.05), and T-cell LCA2 cells (p=0.01), were approximately doubled at the time of dose 5. At the time of dose 10, the T-helper LCA cell levels were decreased by about 50%, (p=0.01). There were no significant changes noted at the last time point, ten days after the last dose was delivered. However, these individual statistical differences did not remain significant after applying the Bonferroni correction for multiple analyses.

### Protocol 2

Effect of Prolonged Melanotan-1 Combined with Sunlight to the Back

This open-label trial in 8 subjects with type 3-4 skin evaluated the effects of a prolonged schedule of MT-1 at 0.16 mg/kg/day for twenty injections (Mon-Fri/week) over 4 weeks. This was combined with full sunlight exposure to one-half of the back for 3-4 days, until a visual tan was apparent. The sunlight exposures were randomized to be given either at the start of MT-1 dosing (n=3), or after ten of the total 20 doses had been administered (n=2). One student subject in the latter group dropped out mid-way through dosing because of time commitments and therefore only two subjects are available for analysis. A control group of three subjects received the same sun exposure regimen to the back without any MT-1 to allow for a comparison of the time to achieve comparable tanning of the exposed hemi-back site.

Table 3 summarises the sun-response characteristics of the subjects in this trial. The mean (SD) sun exposure time required for a visually perceptible tan on the exposed back site in the MT-1 group was 87 (4.5) minutes. This was delivered over a median of three days, with each daily exposure averaging 30 minutes. By comparison, in the sunlight-only control group, a median of five exposures of 25-35 minutes each were required to achieve the same degree of tanning at the exposed back site. The total mean (SD) sun exposure time in this group was 165 (15) minutes, double that in the MT-1 group (p<0.001).

TABLE 3
Characteristics of Subjects Receiving Sunlight
With or Without a Prolonged Course Melanotan-1

| | | | | | Sun Exposure for a | | |
|---|---|---|---|---|---|---|---|
| | | | | | Visually-Apparent Tan | | |
| | | | Skin | MED | | Total | MT-1 |
| No. | Age | Sex | Type | (mjoules) | Days (Minutes) | (Min) | (days) |
| 813 | 34 | M | 3 | 15.75 | 1(30), 2(20), 3(40) | 90 | 1-20 |
| 814 | 24 | M | 3 | 21.0 | 2(20), 3(30), 5(40) | 90 | 1-20 |
| 815 | 25 | M | 4 | 26.25 | 1(15), 2(15), 3(20) | 90 | 1-20 |
| | | | | | 18(20), 10(20) | | |
| 816 | 22 | M | 3 | 15.75 | 13(25), 14(30), 15(25) | 80 | 1-20 |
| 817 | 39 | M | 3 | 21.0 | 13(30), 14(25), 15(30) | 85 | 1-20 |
| 821 | 44 | M | 3 | 21.0 | 1(35), 2(35), 3(30), | 165 | None |
| | | | | | 4(30), 5(35) | | |
| 820 | 32 | M | 3 | 15.75 | 1(35), 2(30), 3(25) | 180 | None |
| | | | | | 4(35), 5(30), 6(25) | | |
| 821 | 23 | M | 4 | 26.25 | 1(30), 2(35), 3(30) | 150 | None |
| | | | | | 4(30), 5(25) | | |

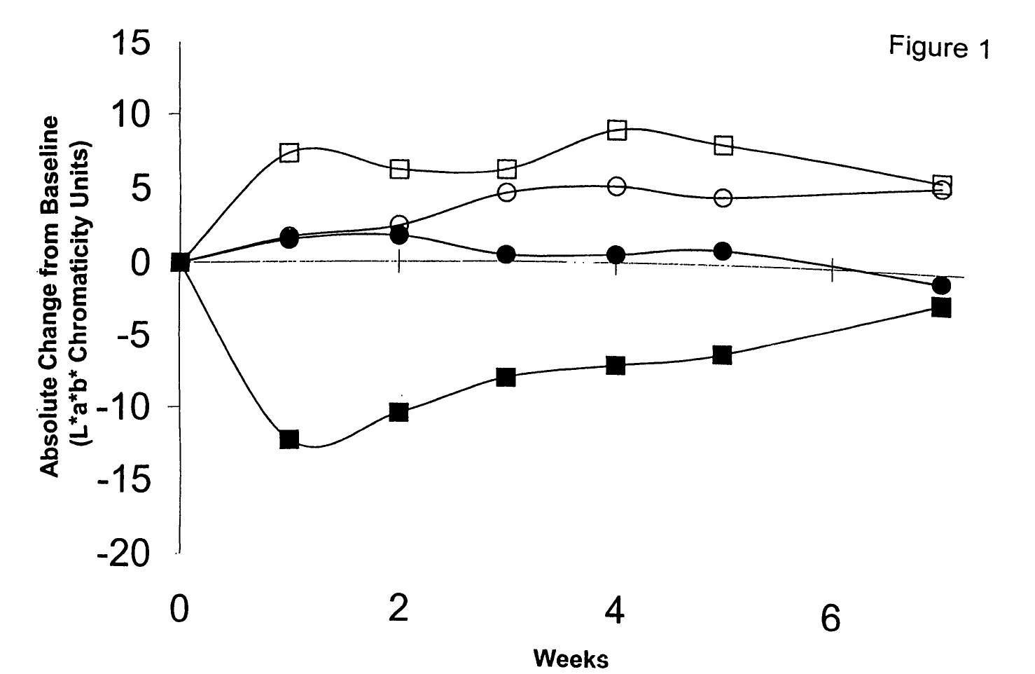

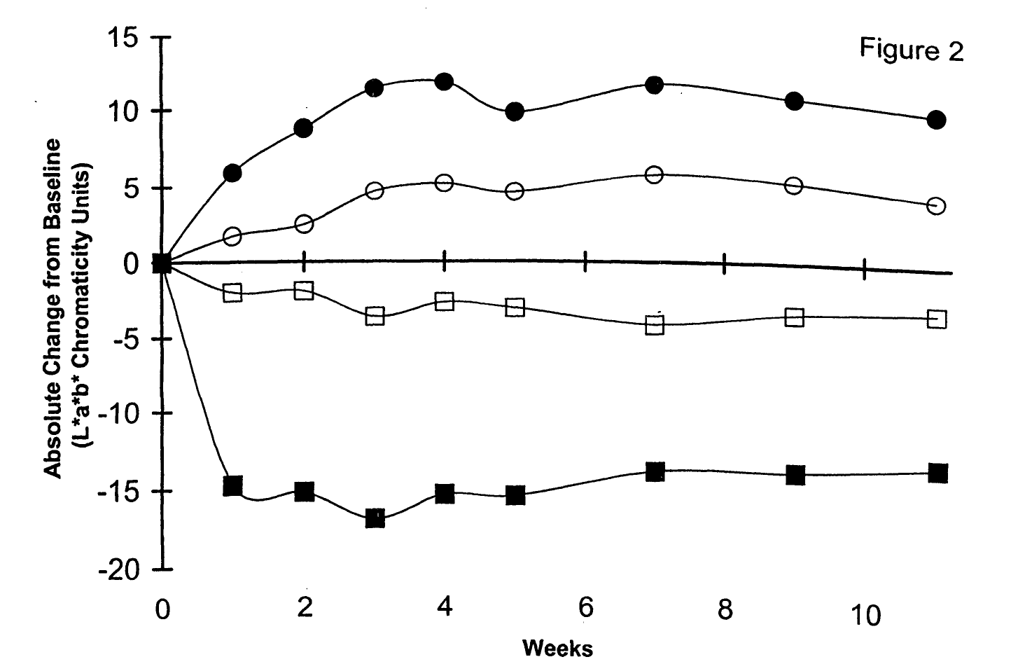

The three control subjects developed significant darkening of the sun-exposed back that involved a mean 7.5 unit decrease in luminance and a 4 unit increase in mean b-values (FIG. 1). As expected, these tans were limited to the sun-exposed site, and the non-exposed back sites actually lightened over the course of the study (FIG. 1, circle symbols,). In addition, the sun-exposed back sites had returned to baseline reflectance values within 5 weeks of sun exposure. FIG. 2 compares the effect of combination sunlight exposure, begun on the first day of MT-1 dosing, with reflectance performed on the subjects adjacent non-sun-exposed back site. This comparison shows that the combination produced rapid and profound skin darkening at the sun-exposed back site (FIG. 2, solid symbols). This was significantly greater than the darkening produced by MT-1 alone at the adjacent (unexposed) back site (FIG. 2, open symbols). The absolute change in reflectance units for the combination was profound involving the largest reflectance changes we have ever recorded: a 10-15 unit decrease in luminance, and a 5-10 unit increase in b-values.

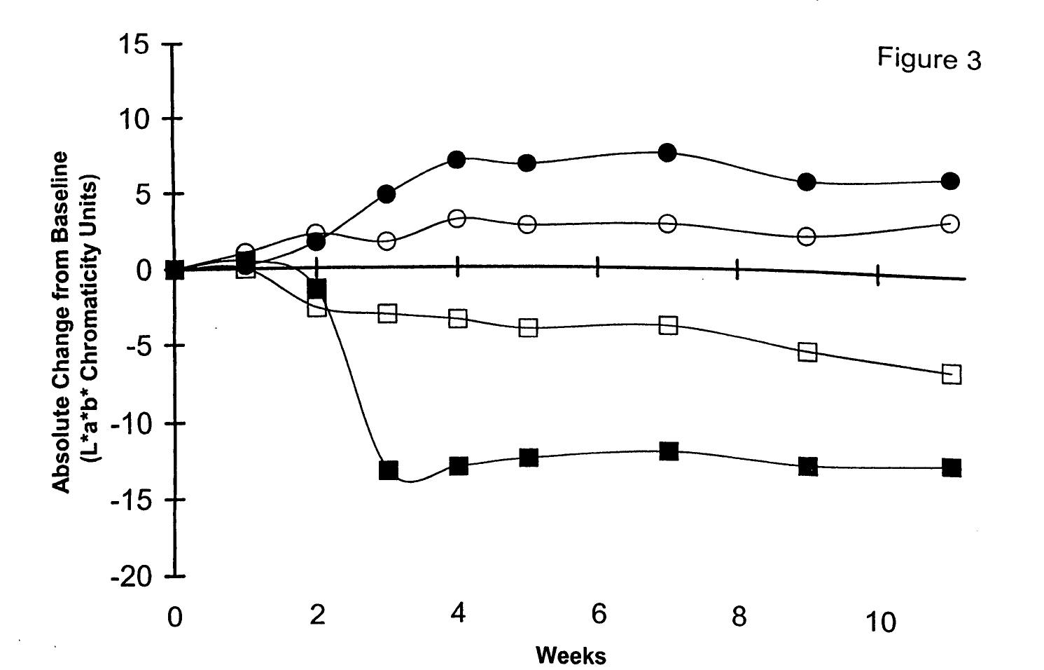

When sun-exposure was added to MT-1 at the end of the first two weeks of dosing, there was a similar increase in darkening at the sun-exposed back site, compared to an adjacent non-sun exposed back site (FIG. 3). However, in this case, the onset of darkening was delayed by 1-2 weeks. This represents a significant difference when compared to sunlight on the first day of MT-1 dosing. FIGS. 2 and 3 show that the duration of darkening was significantly prolonged for all subjects receiving sunlight-plus MT-1. In this case, it did not matter whether sunlight was added at the start or middle of the MT-1 dosing period. Indeed, at the conclusion of reflectance monitoring at 11 weeks, all MT-1 treated subjects still maintained significant tanning of the both sun-exposed and non-exposed back sites. This differs dramatically from the sun-only controls, wherein reflectance values had all returned to baseline after 5 weeks (FIG. 1). The difference is even more remarkable when one considers that the controls received almost twice as much total sun exposure to the back.

Since the group receiving MT-1 in Protocol 2 experienced the greatest cumulative drug exposure to date, the question of side effects has special importance. The most common side effect was facial and upper truncal flushing which occurred variably. There were nine instances of flushing in 3 of the 5 subjects. As in our prior studies, the onset was within minutes of MT-1 injection and it typically resolved with 30-60 minutes. The most serious side effect was nausea, which was experienced by two of the five subjects. This began within 40 minutes of the first injection number in subject 817. To prevent nausea, the next three injections in this subject were given after 10 mg of prochlorperazine (Compazine^{R}), had been administered orally. Another subject, No. 816, also experienced nausea after the second dose and received 10 mg of oral prochlorperazine before the next three doses. Later MT-1 doses were given with no antiemetics and there was no significant nausea. The only other reported side effect was afternoon fatigue or somnolence, which was reported in 3 subjects. For example, during week three, one subject described a two hour period of fatigue after each injection. In the other subject, general fatigue was described throughout the second week of the injections with some persistence over the weekend when no MT-1 was administered. None of these side effects were of moderate or severe intensity and there was no evidence of cumulative toxicity. Indeed, most side effects were reported during the first two weeks of the 4 week regimen, and only one instance each of flushing and fatigue were reported in the last (fourth) week of MT-1 dosing.

Discussion:

The primary goal of the current studies was to characterise the effect of MT-1 combined with UV light. The results show that the synthetic superpotent melanotropin, MT-1, can be safely combined with small amounts of UV-B from a solar simulator, or with brief exposures to full sunlight. The latter combination produced a marked enhancement of skin tanning, with the most rapid onset seen for sunlight added at the start of MT-1 dosing. We have further shown that MT-1 can be administered for 4 weeks at a daily dose of 0.16 mg/kg, without producing cumulative, more intense or different side effects. The 0.16 mg/Kg dose of MT-1 is superior to the 0.08 mg/kg dose used in the original clinical study⁷ in terms of both the degree of tanning, as well as the number of anatomic sites which responded by darkening. For example, in our original study of 0.08 mg/kg in 28 male subjects, significant skin darkening was only observed on the forehead, cheek and neck⁷. In contrast, the results from Protocol 1, show that significant darkening can be achieved at most body sites, including in some cases, the buttocks, wherein melanocortin receptor densities are very low¹⁸. We were also able to demonstrate a significant enhancement at sites receiving concomitant UV-B radiation from the solar simulator. There was one female subject with Type 4 skin by history, who did not respond at any skin site to the 0.16 mg/kg dose of MT-1. This is the first observation of a completely non-responsive individual, and there is no clear explanation at this time.

The native hormone, alpha-MSH, has been reported to have broad anti-inflammatory activities in experimental models of inflammations¹⁰. These effects include inhibition of arthritis in a rat model¹¹, reduction of endotoxin-induced liver inflammation in a septic shock model¹². and improved survival in a model of endotoxemia and peritonitis¹⁰. These effects may be mediated by alpha-MSH-induced inhibition of the synthesis and activity of cytokines and chemo-attractive chemokines in neutrophils¹⁰. Alternatively direct effects on neutrophil migration and superoxide dismutase productions have been reported. Protocol 1 indirectly addressed the issue of immunologic activity for MT-1 in humans receiving ten injections of 0.16 mg/kg. At the end of the two week dosing period, we could not demonstrate any significant changes in the absolute numbers of 17 different white blood cell subtypes in the peripheral blood of 7 of these subjects. However, the effectiveness of these peripheral blood cells to mount an immunologic reaction was not evaluated, and therefore, we cannot rule out an alteration in immune response induced by MT-1. On the other hand, no infections have been observed in any of the approximately 100 normal subjects treated with MT-1 to date^{7,9,13}. Thus, while the question of whether MT-1 induces immunologic alterations in humans is still largely unanswered, we do know that it does not acutely alter the number of several classes of immunologic white blood cells in the peripheral blood. The lack of an immunologic effect for MT-1 is also consistent with a dermal study in mice wherein the native hormone blocked contact hypersensitivity responsiveness, but MT-1 did not²⁰.

The other side effects of Melanotan-1 seen in this study are similar to those previously reported^{7,9,13}. Nausea induced by MT-1 was seen in about 20% of the current subjects and required antiemetic treatment in only two subjects. This effect may be mediated by interaction of MT-1 with melanocortin-3 receptors, (MC3R), which have been found in the gut tissues of animals²¹. The fact that these mild gastrointestinal side effects were infrequent and were not cumulative, suggests that any activation of MC3R by MT-1 is not dose-limiting. The biochemical pathway for the other MT-1 side effects, notably facial flushing and fatigue, are not known. It is unlikely that the acute flushing reactions in the upper trunk are mediated by melanotropic activation of MC1R. The onset of the reactions are rapid, and more time would be required for the synthesis and release of melanin following MT-1 stimulation. Thus, other vasoactive pathways must be involved in mediating this unusual side effect. The only other side effect, fatigue, was also seen in our prior Phase I dose-escalation study, and was dose-dependent in severity¹³. In our current study, fatigue of a mild nature was noted at some time in about one-third of the subjects. Like the other toxicities, it did not recur with each dose and was not cumulative in intensity when it did recur. Whether these effects are mediated by binding to MC3R and MC4R, found in the brain²², is not known. This is unlikely however, since a prior (unpublished) pharmacokinetic study could not detect significant drug uptake into the brain in rats given radiolabelled MT-1.

Perhaps the most important observation in the two clinical studies of MT-1 and light, is the observation of marked tanning synergy with the combination of UV-B-light (Protocol 1) and sunlight Protocol 2). The degree of skin darkening measured at both light exposed sites was significantly greater than that achieved with either light, or drug alone. Indeed, the tanning observed in the sun-exposed back in Protocol 2 is the most intense we have ever measured. Furthermore, the combination of MT-1 plus sunlight produced a long lasting tan at the sun-exposed back sites. This had still not returned to baseline reflectance values 11 weeks after MT-1 dosing started, significantly longer than we have seen previously using a two week course of MT-1 at the 0.16 mg/kg dose¹³. The 4 week course of MT-1 used in Protocol 2 also represents the largest cumulative exposure to drug to date. Importantly, we saw no new side effects or more intense side effects with this doubled exposure to MT-1.

Melanotan-1 has high binding affinity for melanocortin 1 receptors, (MC1R) in the epidermis¹. Possibly due to its high potency in experimental systems, it can activate receptors that have mutations at various sites in the seven transmembrane domains of the molecule, a feature not shared with natural alpha-MSH²³. This may have important implications for individuals with MC1R gene mutations since these persons tan poorly and are at a higher risk for both basal and squamous cell carcinomas. In addition, other studies suggest that the risk of melanoma is also increased in individuals with MC1R variant alleles⁶. Thus, the availability of a more potent agonist for the MC1R, raises the possibility that MT-1 could induce a protective tan even in individuals with mutated receptor genes. While this might reduce the risk for all types of sun-induced skin cancers, the current studies did not test this hypothesis. Rather, the subjects in the three current protocols were selected for the ability to respond to sunlight by tanning without burning (Fitzpatrick skin types³⁻⁴). Therefore, we do not know whether humans with MC1R gene mutations will respond to MT-1.

### REFERENCES

1. Hadley M E. The melanotropic hormones. In: Brake D, editor. Endocrinology. 4th Edition, Simon & Schuster; (1982). p. 153-76.
2. Thody A J, Higgins E M, Wakamatsu K, Ito S, Burchill S A, Marks J M. Pheomelanin as well as eumelanin are present in human epidermis. J. Invest Dermatol. (1991); 97:340-44.
3. Fitzpatrick T B. The validity and practicality of sun-reactive skin types I through VI. Arch Dermatol (1988); 124:869-71.
4. Valverde P, Healy E, Jackson I, Rees J L, Thody A J. Variants of the melanocyte-stimulating hormone receptor gene are associated with red hair and fair skin in humans. Nature Genet (1995); 11:328-30.
5. Box N F, Duffy D L, Irving R E, Russell A, Chen W, Griffiths L R, et al. Melanocortin-1 receptor genotype is a risk factor for basal and squamous cell carcinoma. J Invest Derm (2001); 116:224-29.
6. Palmer J S, Duffy D L, Box N F, Aitken J F, O'Gorman L E, Green A C, et al. Melanocortin-1 receptor polymorphisms and risk of melanoma: is the association explained solely by pigmentation phenotype? Am J Hum Genet (2000); 66:176-86.
7. Levine N, Sheftel S N, Eytan T, Doff R T, Hadley M E, Weinrach J C, et al. Induction of skin tanning by subcutaneous administration of a potent synthetic melanotropin. JAMA (1991); 266:2730-736.
8. Sawyer T K, Sanfilippo V J, Hruby V J et al. [Nle⁴-D-Phe⁷)-α-melanocyte stimulating hormones: a highly potent α-melanotropin with ultralong biological activity. Proc Natl Acad Sci USA (1980); 77:5754-8.
9. Dorr R T, Dvorakova K, Brooks C, Lines R, Levine N, Schram K, et al. Increased eumelanin expression and tanning is induced by a superpotent melanotropin [Nle⁴D-Phe⁷]-α-MSH in humans. Photochem Photobiol (2000); 72:526-32.
10. Lipton J M, Ceriani G, Macaluso A, McCoy D, Cames K, Biltz J, et al. Antiinflammatory effects of the neuropeptide alpha-MSH in acute, chronic and systemic inflammation. Ann NY Acad Sci (1994); 741:137-48.
11. Ceriani G, Diaz J. Murphree S, Catania A, Lipton J M. The neuropeptide alpha-melanocyte-stimulating hormone inhibits experimental arthritis in rats. Neuroimmunomodulation (1994); 1:28-32.
12. Chiao H S, Foster R, Thomas J, Lipton J M, Star R A. α-melanocyte-stimulating hormone reduces endotoxin-induced liver inflammation. J Clin Invest (1996); 97:2038-44.
13. Levine N, Dorr R T, Ertl G A, Brooks C, Alberts D S. Effects of a potent synthetic melanotropin, Nle⁴-D-Phe⁷-α-MSH (Melanotan-1) on tanning: a dose-ranging study. J Derm Treat (1999); 19:127-32.
14. Sullivan J D Jr, Valois F W, Watson S W. Endotoxins: the Limulus amebocyte lysate system. In: Bernheimer A W, ed. Mechanisms of Bacterial Toxicology. New York, N.Y.: John Wiley & Sons Inc. (1976). p. 217-220.
15. Porgess S B, Kaidbey K H, Grove G L. Quantification of visible light-induced melanogenesis in human skin. Photoderm (1988); 5:197-200.
16. Westerhof W, van Hasselt B M M, Kammeijer A. Quantification of UV-induced erythema with a portable computer controlled chromometer. Photoderm (1986); 3:310-314.
17. Seitz J C, Whitmore C G. Measurements of erythematous and tanning responses in human skin using a tri-stimulus colorimeter. Dermatol (1988); 177:70-5.
18. Szabo G. The number of melanocytes in human epidermis. BMJ. (1954); 1:1016-17;
19. Van Epps D E, Mason MM Comparative Aspects neuropeptide. Function (1991).
20. Rheins L A, Cotleur A L, Kleier R S, Hoppenjans W B, Saunder D N., Nordhund J & J. Alpha-melanocyte stimulating hormone modulates contact hypersensitivity responsiveness in C57/BL6 mice. J Invest Dermatol (1989); 93:511-17.
21. Mountjoy K G, Ribbins L S, Mortrud M T, Cone R C. The cloning of a family of genes that encode the melano-cortin receptors. Science; 257:543-46.
22. Mountjoy K G, Mortrud M T, Low M J, Simerly R B, Cone R D. Localization of the melanocortin-4 receptor (MC4-R) in neuroendocrine and autonomic control circuits in the brain. Mol Endocrin (1994); 8:1298-1308.
23. Yang Y-K, Dickinson C, Haskell-Luevano C, Gantz I. Molecular basis for the interaction of [Nle⁴-D-Phe⁷]-melanocyte stimulating hormone with the human melanocortin-1 receptor (melanocyte α-MSH receptor). J Biol Chem (1997); 272:23000-10.
24. Bhardwaj R, Hadley M E, Dorr R T, Dvorakova K, Brooks C, Blanchard J. Pharmacologic response of a controlled-release PLGA formulation for the alpha-melanocyte stimulating hormone analog, Melanotan-1. Pharmaceutical Research (2000); 17:583-9.
25. Sawyer T K, Hruby V J, Darman P S, Hadley M E. [4-Half-Cystine, 10-Half-Cystine]-α-Melanocyte Stimulating Hormone: A cyclic α-melanotropin exhibiting superagonist biological activity. Proc. Natl. Acad. Sci. USA, 79, 1751-1755 (1982).
26. Knittel J J, Sawyer T K, Hruby V J, Hadley M E. Structure-activity studies of highly potent [Cys⁴,Cys¹⁰]melanotropin analogues. J. Med. Chem. 28, 125-129 (1983).

1. A method for the stimulation of integumental melanocytes in a mammal, which comprises the steps of:
(i) administering to said mammal an amount of alpha-MSH or an alpha-MSH analogue effective to stimulate melanocytes in the skin or other epidermal tissue; and
(ii) exposing said skin or other epidermal tissue to ultraviolet (UV) irradiation.

2. A method for stimulating melanin production in a mammal, which comprises the steps of:
(i) administering to said mammal an amount of alpha-MSH or an alpha-MSH analogue effective to stimulate melanin production in the skin or other epidermal tissue; and
(ii) exposing said skin or other epidermal tissue to ultraviolet (UV) irradiation.

3. A method for inducing tanning in a mammal, which comprises the steps of:
(i) administering to said mammal an amount of alpha-MSH or an alpha-MSH analogue effective to induce tanning in the skin or other epidermal tissue; and
(ii) exposing said skin or other epidermal tissue to ultraviolet (UV) irradiation.

4. The method according to claim 1, wherein said mammal is a human.

5. A method for inducing skin tanning in a human which comprises the steps of:
(i) administering to said human an amount of alpha-MSH or an alpha-MSH analogue effective to induce tanning in the skin or other epidermal tissue; and
(ii) exposing said skin or other epidermal tissue to ultraviolet (UV) irradiation.

6. The method according to claim 1, wherein the alpha-MSH analogue is a compound of the formula:
R₁-W-X-Y-Z-R₂
wherein
R₁ is selected from the group consisting of Ac-Gly-, Ac-Met-Glu-, Ac-Nle-Glu-, and Ac-Tyr-Glu-;
W is selected from the group consisting of -His-and -D-His-;
X is selected from the group consisting of -Phe-, -D-Phe-, -Tyr-, -D-Tyr-, and -(pNO₂)D-Phe⁷-;
Y is selected from the group consisting of -Arg- and -D-Arg-;
Z is selected from the group consisting of -Trp- and -D-Trp-; and
R₂ is selected from the group consisting of —NH₂; -Gly—NH₂; and -Gly-Lys—NH₂.

7. The method according to claim 1, wherein the alpha-MSH analogue is a compound selected from the group consisting of:
[D-Phe⁷]-alpha-MSH
[Nle⁴, D-Phe⁷]-alpha-MSH
[D-Ser¹, D-Phe⁷]-alpha-MSH
[D-Tyr², D-Phe⁷]-alpha-MSH
[D-Ser³, D-Phe⁷]-alpha-MSH
[D-Met⁴, D-Phe⁷]-alpha-MSH
[D-Glu⁵, D-Phe⁷]-alpha-MSH
[D-His⁶, D-Phe⁷]-alpha-MSH
[D-Phe⁷, D-Arg⁷]-alpha-MSH
[D-Phe⁷, D-Trp⁹]-alpha-MSH
[D-Phe⁷, D-Lys¹¹]-alpha-MSH
[D-Phe[[-]]⁷, D-Pro¹²]-alpha-MSH
[D-Phe⁷, D-Val¹³]-alpha-MSH
[D-Ser¹, Nle⁴, D-Phe⁷]-alpha-MSH
[D-Tyr², Nle⁴, D-Phe⁷]-alpha-MSH
[D-Ser³, Nle⁴, D-Phe⁷]-alpha-MSH
[Nle⁴, D-Glu⁵, D-Phe⁷]-alpha-MSH
[Nle⁴, D-His⁶, D-Phe⁷]-alpha-MSH
[Nle⁴, D-Phe⁷, D-Arg⁸]-alpha-MSH
[Nle⁴, D-Phe⁷, D-Trp⁹]-alpha-MSH
[Nle⁴, D-Phe⁷, D-Lys¹¹]-alpha-MSH
[Nle⁴, D-Phe⁷, D-Pro¹²]-alpha-MSH
[Nle⁴, D-Phe⁷, D-Val¹³]-alpha-MSH
c[Cys⁴, Cys¹⁰]-alpha-MSH
c[Cys⁴, D-Phe⁷, Cys¹⁰]-alpha-MSH
c[Cys⁴, Cys¹¹]-alpha-MSH
c[Cys⁵, Cys¹⁰]-alpha-MSH
c[Cys⁵, Cys¹¹]-alpha-MSH
c[Cys⁴, Cys¹⁰]-alpha-MSH₄₋₁₃
c[Cys⁴, Cys¹⁰]-alpha-MSH₄₋₁₂
[Nle⁴, D-Phe⁷]-alpha-MSH₄₋₁₀
[Nle⁴, D-Phe⁷]-alpha-MSH₄₋₁₁
[D-Phe⁷]-alpha-MSH₅₋₁₁
[Nle⁴, D-Tyr⁷]-alpha-MSH₄₋₁₁
[(pNO₂)D-Phe⁷]-alpha-MSH₄₋₁₁
[Tyr⁴, D-Phe⁷]-alpha-MSH₄₋₁₀
[Tyr⁴, D-Phe⁷]-alpha-MSH₄₋₁₁
[Nle⁴]-alpha-MSH₄₋₁₁
[Nle⁴, (pNO₂)D-Phe⁷]-alpha-MSH₄₋₁₁
[Nle⁴, D-His⁶]-alpha-MSH₄₋₁₁
[Nle⁴, D-His⁶, D-Phe⁷]-alpha-MSH₄₋₁₁
[Nle⁴, D-Arg⁸]-alpha-MSH₄₋₁₁
[Nle⁴, D-Trp⁹]-alpha-MSH₄₋₁₁
[Nle⁴, D-Phe⁷, D-Trp⁹]-alpha-MSH₄₋₁₁
[Nle⁴, D-Phe⁷]-alpha-MSH₄₋₉ and
[Nle⁴, D-Phe⁷, D-Trp⁹]-alpha-MSH₄₋₉

8. The method according to claim 7, wherein the alpha-MSH analogue is a compound selected from the group consisting of:
[Nle⁴, D-Phe⁷]-alpha-MSH
[Nle⁴, D-Phe⁷]-alpha-MSH₄₋₁₀
[Nle⁴, D-Phe⁷]-alpha-MSH₄₋₁₁
[Nle⁴, D-Phe⁷, D-Trp⁹]-alpha-MSH₄₋₁₁ and
[Nle⁴, D-Phe⁷]-alpha-MSH₄₋₉

9. The method according to claim 8, wherein the alpha-MSH analogue is [Nle⁴, D-Phe⁷]-alpha-MSH.

10. The method according to claim 1, wherein said step of exposing to UV irradiation is carried out subsequent to said step of administration of alpha-MSH or an alpha-MSH analogue.

11. The method according to claim 1, wherein said administration is oral, parenteral or transdermal administration.

12. The method according to claim 1, wherein said ultraviolet (UV) irradiation consists essentially of or comprises UV-B irradiation.

13-17. (canceled)
